(19) United States
(12) Patent Application Publication
(10) Pub. No.: US 2026/0240800 A1
(43) Pub. Date: Aug. 20, 2026

(54) ELAFIBRANOR FOR THE TREATMENT OF PRIMARY BILIARY CHOLANGITIS

(71) Applicant: Genfit, Loos (FR)

(72) Inventors: Pascal Birman, Paris (FR); Alice Roudot, Lomme (FR); David Magrez, Lambersart (FR)

(21) Appl. No.: 19/647,808

(22) Filed: Apr. 14, 2026

Related U.S. Application Data
(63) Continuation of application No. PCT/EP2024/080132, filed on Oct. 24, 2024.

(30) Foreign Application Priority Data
Oct. 25, 2023 (EP) 23306863.4
Oct. 14, 2024 (EP) 24306685.9

(51) Int. Cl. A61K 31/192; A61P 1/16

(52) U.S. Cl. CPC A61K 31/192; A61P 1/16

(57) ABSTRACT

The present invention relates to a pharmaceutical composition comprising a compound selected from the group consisting of elafibranor, 2-[2,6-dimethyl-4-[3-[4-(methylthio)phenyl]-3-oxo-propyl]phenoxy]-2-methylpropanoic acid (GFT1007), and a pharmaceutically acceptable salt of elafibranor or GFT1007, for use in a method for treating primary biliary cholangitis (PBC) in a subject having PBC with a baseline plasma alkaline phosphatase (ALP) level of more than 1.67×ULN and at most 3×ULN.

### RELATED APPLICATIONS

This application is a Continuation of International Application No. PCT/EP2024/080132, filed Oct. 24, 2024, which claims the benefit of and priority to European Application No. 23306863.4, filed Oct. 25, 2023. The entire teachings of each of the above applications are incorporated herein by reference. International Application No. PCT/EP2024/080132 was published under PCT Article 21 (2) in English.

### TECHNICAL FIELD

The present invention relates to the field of medicine, in particular the treatment of cholestatic diseases, and more specifically PBC

### BACKGROUND

Primary biliary cholangitis (PBC) is a rare, chronic, progressive, cholestatic liver disease of autoimmune etiology, characterized by injury of the intrahepatic bile ducts that, in untreated patients, may progress to hepatic fibrosis, cirrhosis, hepatic decompensation, and death unless patients receive a liver transplant. PBC disproportionately affects women vs men (approximately 10:1) and is typically diagnosed in patients between 40 years to 60 years of age. In Europe, North America, Asia, and Australia, the incidence and prevalence rates of PBC are reported as ranging from 0.33 to 5.8 per 100,000 inhabitants and 1.91 to 40.2 per 100,000 inhabitants, respectively.

Over 60% of the newly diagnosed cases are asymptomatic. The majority of asymptomatic patients become symptomatic within 10 years. The most common symptoms of PBC are generalized fatigue (in 70% of cases) and pruritus (Crosignani A, et al., Clinical features and management of primary biliary cirrhosis. World J Gastroenterol. 2008; 14 (21): 3313-3327). The mechanisms underlying these symptoms are not well elucidated and neither correlates with disease stage or clinical outcomes.

PBC represents one of the leading indications for liver transplantation. Despite its rarity, PBC remains therefore an important cause of morbidity in the Western world. PBC has also been identified as an important risk factor for hepatocellular carcinoma.

PBC is characterized by cholestasis caused by autoimmune destruction of biliary ductules with progressive impairment of bile flow in the liver. This results in increased hepatocellular bile acid concentrations which are toxic to the liver. Such hepatocellular injury is associated with a local inflammatory response resulting early on in an abnormal elevation of serum alkaline phosphatase (ALP) levels. Therefore, biologically, PBC is manifested by an elevation of ALP levels of more than 1.5 times the upper limit of normal (ULN). The ALP levels are generally associated with disease progression. Indeed, elevations in ALP level are associated with a risk of liver transplantation or death that is 2.0 to 2.5 times higher than the risk associated with normal levels. As shown in an international follow-up study, ALP>2×ULN is associated with a 2.2× greater risk for liver transplant or death at 1 year compared to those who remained ≤2×ULN (P<0.0001) (Lammers W J et al, Gastroenterology, 2014; 147 (6): 1338)

An abnormally elevated bilirubin level, which occurs later in disease progression, is also a strong predictor of outcomes, with a risk of liver transplantation or death that was 5.1 to 10.7 times the risk associated with normal levels.

In October 2023, the only approved drugs to treat patients with PBC were ursodeoxycholic acid (UDCA) and Ocaliva® (obeticholic acid, OCA).

UDCA (Ursodeoxycholic acid) has been shown to improve ALP and bilirubin levels, and to delay histological progression, thereby increasing liver transplant-free survival. However, up to 40% of UDCA-treated patients have a suboptimal response (Ali A H, et al., Orphan drugs in development for primary biliary cirrhosis: challenges and progress. Orphan Drugs: Research and Reviews. 2015; 5:83-97). ALP has been shown to remain elevated in up to 70% of patients who are currently being treated or are intolerant to UDCA (Lammers W J, et al., Levels of alkaline phosphatase and bilirubin are surrogate end points of outcomes of patients with primary biliary cirrhosis: an international follow-up study. Gastroenterology. 2014; 147 (6): 1338-1349.). Such patients remain at risk of disease progression and longer term adverse clinical outcomes. At present, 44% of UDCA-treated patients are progressing to liver transplant or death over 15 years.

Obeticholic acid (OCA) (Ocaliva®), which has been shown to reduce ALP, has been recently approved in several countries as second line therapy for the treatment of PBC as monotherapy in adults unable to tolerate UDCA or in combination with UDCA in adults with an inadequate response to UDCA. A decrease in ALP levels is recognized as a particularly relevant surrogate marker for the treatment of PBC, and was recently used as the basis for conditional market approval of OCA in this indication.

Considering the efficacy and tolerability issues with the current treatment options available, there is an unmet need for therapeutic options for patients with PBC, allowing a significant reduction in plasma ALP levels.

Elafibranor (2-(2,6-dimethyl-4-{3-[4-(methylsulfanyl)phenyl]-3-oxopropen-1-yl}phenoxy)-2-methylpropanoic acid) was evaluated in a phase 3 study for the treatment of PBC (ELATIVE®). The results of phase 3 on PBC show that the mean relative change (%) from baseline to endpoint in serum ALP was-48.3% for the elafibranor 80 mg treatment group, and 3.2% for placebo.

Thus, the treatment with elafibranor resulted in a consistent, statistically significant reduction in plasma ALP levels from baseline when compared to placebo. Moreover, elafibranor is safe and well-tolerated by the patients.

### SUMMARY OF THE INVENTION

The phase 3 clinical study has surprisingly shown that the treatment of participants with 2-(2,6-dimethyl-4-{3-[4-(methylsulfanyl)phenyl]-3-oxoprop-1-en-1-yl}phenoxy)-2-methylpropanoic acid (elafibranor, formerly called GFT505) (80 mg daily) provides a better efficacy for participants having PBC with a baseline plasma ALP level of more than 1.67×ULN and at most 3×ULN than for participants having PBC with a baseline plasma ALP level of more than 3×ULN. The efficacy of elafibranor is more than 3 times greater for subjects having PBC with a baseline plasma ALP level of more than 1.67×ULN and at most 3×ULN than for subjects having PBC with a baseline plasma ALP level of more than 3×ULN.

The present invention thus relates to a pharmaceutical composition comprising a compound selected from the group consisting of elafibranor, 2-[2,6-dimethyl-4-[3-[4-(methylthio)phenyl]-3-oxo-propyl]phenoxy]-2-methylpropanoic acid (GFT1007), and a pharmaceutically acceptable salt of elafibranor or GFT1007, for use in a method for treating primary biliary cholangitis (PBC) in a subject having PBC with a baseline plasma alkaline phosphatase (ALP) level of more than 1.67×ULN and at most 3×ULN.

The present invention also relates to a pharmaceutical composition comprising a compound selected from the group consisting of elafibranor, GFT1007, and a pharmaceutically acceptable salt of elafibranor or GFT1007, for use in a method for reducing baseline plasma ALP level in a subject having PBC with a baseline plasma ALP level of more than 1.67×ULN and at most 3×ULN.

The invention further relates to the use of a pharmaceutical composition comprising a compound selected from the group consisting of elafibranor, 2-[2,6-dimethyl-4-[3-[4-(methylthio)phenyl]-3-oxo-propyl]phenoxy]-2-methylpropanoic acid (GFT1007), and a pharmaceutically acceptable salt of elafibranor or GFT1007, for the manufacture of a medicament for use in a method for treating primary biliary cholangitis (PBC) in a subject having PBC with a baseline plasma alkaline phosphatase (ALP) level of more than 1.67×ULN and at most 3×ULN.

The present invention further relates to the use of a pharmaceutical composition comprising a compound selected from the group consisting of elafibranor, GFT1007, and a pharmaceutically acceptable salt of elafibranor or GFT1007, for the manufacture of a medicament for use in a method for reducing baseline plasma ALP level in a subject having PBC with a baseline plasma ALP level of more than 1.67×ULN and at most 3×ULN.

The present invention also relates to a method for treating primary biliary cholangitis (PBC) in a subject having PBC with a baseline plasma alkaline phosphatase (ALP) level of more than 1.67×ULN and at most 3×ULN, comprising administering to said subject, a therapeutic effective amount of a pharmaceutical composition comprising a compound selected from the group consisting of elafibranor, 2-[2,6-dimethyl-4-[3-[4-(methylthio)phenyl]-3-oxo-propyl]phenoxy]-2-methylpropanoic acid (GFT1007), and a pharmaceutically acceptable salt of elafibranor or GFT1007.

The present invention also relates to a method for reducing baseline plasma ALP level in a subject having PBC with a baseline plasma ALP level of more than 1.67×ULN and at most 3×ULN, comprising administering to said subject, a therapeutic effective amount of a pharmaceutical composition comprising a compound selected from the group consisting of elafibranor, GFT1007, and a pharmaceutically acceptable salt of elafibranor or GFT1007.

### DESCRIPTION OF THE FIGURES

FIG. 1: Normalization of ALP

FIG. 1 shows that complete normalization of alkaline phosphatase at Week 52 occurs in 15% and 0% of participants receiving elafibranor and placebo, respectively (difference, 15%; odds ratio, infinity; 95% CI, 2.8 to infinity; p=0.0019).

FIG. 2: Reductions of ALP levels

FIG. 2 shows rapid reductions in alkaline phosphatase levels observed from baseline through Week 4 in the elafibranor group, and sustained at levels significantly lower than the placebo group through Week 52.

FIG. 3 shows the percentage of participants receiving elafibranor with a biochemical response at Week 52 stratified by baseline ALP levels (≤2, 2-≤2.5, 2.5-≤3, 3-≤4, and >4×ULN). Biochemical response (the primary endpoint) was defined as ALP<1.67×ULN, with a ≥15% reduction from baseline, and normal TB.

FIG. 4 shows the percentage of participants receiving elafibranor with ALP normalization at Week 52 stratified by baseline ALP levels (≤2, 2-≤2.5, 2.5-≤3, 3-≤4, and >4×ULN).

FIG. 5 shows the mean percentage change from baseline to Week 52 in ALP stratified by baseline ALP levels (≤2, 2-≤2.5, 2.5-≤3, 3-≤4, and >4×ULN).

### DETAILED DESCRIPTION OF THE INVENTION

The present invention relates to a pharmaceutical composition comprising a compound selected from the group consisting of elafibranor, 2-[2,6-dimethyl-4-[3-[4-(methylthio)phenyl]-3-oxo-propyl]phenoxy]-2-methylpropanoic acid (GFT1007), and a pharmaceutically acceptable salt of elafibranor or GFT1007, for use in a method for treating primary biliary cholangitis (PBC) in a subject having PBC with a baseline plasma alkaline phosphatase (ALP) level of more than 1.67×ULN and at most 3×ULN.

In a particular embodiment, the pharmaceutical composition of the invention can be used for normalizing ALP, albumin and/or bilirubin level(s) in a subject having PBC with a baseline plasma ALP level of more than 1.67×ULN and at most 3×ULN.

In a particular embodiment, the subject having PBC has a baseline plasma ALP level of more than 1.67×ULN and at most 2.5×ULN.

In another particular embodiment, the subject having PBC has a baseline plasma ALP level of more than 1.67×ULN and at most 2×ULN.

In the context of the invention, the terms “baseline plasma level” refer to the plasma level of a subject before starting any treatment with the composition of the invention. For example, “baseline ALP plasma level” refers to the ALP plasma level of a subject before starting any treatment with the composition of the invention.

In a particular embodiment, the treatment results in a level of ALP lower than 1.67×ULN (upper limit of normal) and optionally total bilirubin within normal limit. The reference range of total bilirubin is 0.2-1.2 mg/dL. The reference range of direct bilirubin is 0.1-0.4 mg/dL.

In a particular variant of this embodiment, the composition of the invention is administered to the subject for decreasing ALP level by at least 15%, preferably by at least 40%.

In a particular embodiment, the composition of the invention is administered to the subject for improving bile acids level such as chenodeoxycholic acid (CDCA), cholic acid, litocholic acid and deoxycholic acid (DCA) levels.

In a further embodiment, the composition of the invention is administered to the subject for improving Paris I, Paris II, Toronto I, Toronto II or UK-PBC risk score.

In another embodiment, the composition of the invention is administered to the subject for:
improving AST, γGT, 5′-nucleotidase, total bilirubin, conjugated bilirubin, ALT and albumin levels;
improving lipid parameters
improving C4 and/or FGF19 levels
improving IgM levels; and
improving 5D-itch scale, PBC 40 QOL, VAS.

In a particular embodiment of the invention, the subject having PBC has a baseline ALP level of more than 1.67×ULN and at most 3×ULN, a baseline AST level of less than 2×ULN and a baseline total bilirubin level (TB) of at most 1 mg/dL.

The term “treatment” or “treating” refers to therapy, prevention, or prophylaxis of a cholestatic disease in a subject in need thereof. The treatment involves the administration of elafibranor, GFT1007 or a pharmaceutically acceptable salt thereof (such as via the administration of a pharmaceutical composition comprising elafibranor) to a subject (e.g. a patient) having a declared disease to prevent, cure, delay, reverse, or slow down the progression of the disease, improving thereby the condition of patients. A treatment may be also administered to subjects that are either healthy or at risk of developing a cholestatic disease.

The term “patient”, “subject”, “individual”, or “participants” are interchangeable and refer to a mammal and more particularly a human including adult and child. The subjects to be treated according to the invention can be appropriately selected on the basis of several criteria associated with cholestatic pathological processes such as previous and/or present drug treatments, associated pathologies, genotype, exposure to risk factors, as well as any other relevant biomarker that can be evaluated by means of any suitable immunological, biochemical, or enzymatic method. The subject to be treated is with PBC, as characterized as follows:
the presence of at least 2 of the following 3 diagnostic factors:
   (i) history of elevated ALP levels for at least 6 months prior to Day 0 (randomization visit)
   (ii) positive Anti-Mitochondrial Antibodies (AMA) titers (> 1/40 on immunofluorescence or M2 positive by enzyme-linked immunosorbent assay (ELISA) or positive PBC-specific antinuclear antibodies
   (iii) liver biopsy consistent with PBC

$$- 1.67 < ULN < ALP \leq 3 \times ULN$$

optionally, taking UDCA for at least 12 months (stable dose for ≥6 months) prior to screening visit.

The present invention further relates to a pharmaceutical composition comprising a compound selected from the group consisting of elafibranor, GFT1007, and a pharmaceutically acceptable salt of elafibranor or GFT1007, for use in a method for reducing baseline plasma ALP level in a subject having PBC with a baseline plasma ALP level of more than 1.67×ULN and at most 3×ULN.

In a particular embodiment, the subject having PBC has a baseline plasma ALP level of more than 1.67×ULN and at most 2.5×ULN.

In another particular embodiment, the subject having PBC has a baseline plasma ALP level of more than 1.67×ULN and at most 2×ULN.

In the context of the invention, the terms “reducing baseline plasma ALP level in a subject” means either a reduction of at least 15% of the baseline plasma ALP level of said subject or a reduction of the ALP level of the subject to no more than 1.67×ULN, after 52 weeks of treatment with the composition of the invention. Reducing baseline plasma ALP level in a subject having PBC helps to improve the condition of said subject, in particular signing a reduction in cholestasis.

In the context of the invention, ALP results are reported in international units per liter (IU/L or U/L). For males older than age 18, the upper limit of normal (ULN) related to the ALP level is 129 U/L. For females older than age 18, the upper limit of normal (ULN) related to the ALP level is 104 U/L.

In a particular embodiment of the invention, the subject to be treated is identified by testing the ALP level of a potential subject and then selecting the subject having baseline plasma ALP level of more than 1.67×ULN and at most 3×ULN. In a particular embodiment, the selected subject has a baseline plasma ALP level of more than 1.67×ULN and at most 2.5×ULN or of more than 1.67×ULN and at most 2×ULN.

The ALP level is testing using methods commonly known in the art. An ALP test requires a blood sample from a vein.

In the context of the invention, AST results are reported in international units per liter (IU/L or U/L). For males older than age 18, the upper limit of normal (ULN) related to the AST level is 40 U/L. For females older than age 18, the upper limit of normal (ULN) related to the AST level is 32 U/L.

In some embodiments of the invention, GFT1007, the active metabolite of elafibranor, is used. GFT1007 is 2-[2,6-dimethyl-4-[3-[4-(methylthio)phenyl]-3-oxo-propyl]phenoxy]-2-methylpropanoic acid. Its properties and synthesis were described in PCT application WO2007/147879, where it is referred to as compound 1.

According to the present invention, the pharmaceutical composition of the invention may include a stereoisomer of elafibranor, of GFT1007, or of a pharmaceutically acceptable salt of elafibranor or of GFT1007.

A stereoisomer is an isomeric compound that has the same molecular formula and sequence of bonded atoms, but differs in the 3D-dimensional orientations of its atoms in space. The stereoisomers include enantiomers, diastereoisomers, cis-trans and E-Z isomers, conformers and tautomers.

Elafibranor or GFT1007 can be formulated as pharmaceutically acceptable salt, particularly an acid or base salt compatible with pharmaceutical use. Salts of elafibranor or GFT1007 implemented herein include pharmaceutically acceptable acid addition salts, pharmaceutically acceptable base addition salts, pharmaceutically acceptable metal salts, ammonium and alkylated ammonium salts. These salts can be obtained during the final purification step of the compound or by incorporating the salt into the previously purified compound.

In particular, “pharmaceutically acceptable salts” include inorganic as well as organic acids salts. Counter-ions may be selected from the following the non-exhaustive list: ammonia, L-arginine, benethamine, benzathine, tert-butylamine (erbumine), calcium hydroxide, choline hydroxide, deanol, diethanolamine (2,2′-iminobis(ethanol), diethylamine, epolamine (1-(2-hydroxyethyl) pyrrolidine), 2-(diethylamino)-ethanol, ethanolamine (2-aminoethanol), ethylenediamine, glycine, hydrabamine, 1H-imidazole, L-Lysine, magnesium hydroxide, meglumine (N-methyl-glucamine), 4-(2-hydroxyethyl)-morpholine, piperazine, potassium hydroxide, sodium hydroxide, triethanolamine (2,2′,2″-nitrilo-tris(ethanol)), tromethamine, zinc hydroxide, in particular tromethamine, potassium, sodium, benethamine, benzathine, L-arginine, ethanolamine, meglumine, glycine, erbumine, L-lysine, epolamine, choline, preferably tromethamine, potassium, sodium, benethamine, benzathine, L-arginine, more preferably tromethamine, potassium, sodium, L-arginine, more particularly tromethamine.

In particular embodiments, the invention implements an ammonia, L-arginine, benethamine, benzathine, tert-butylamine (erbumine), calcium, choline, deanol, diethanolamine (2,2′-iminobis(ethanol), diethylamine, epolamine (1-(2-hydroxyethyl) pyrrolidine), 2-(diethylamino)-ethanol, ethanolamine (2-aminoethanol), ethylenediamine, glycine, hydrabamine, 1H-imidazole, L-Lysine, magnesium, meglumine (N-methyl-glucamine), 4-(2-hydroxyethyl)-morpholine, piperazine, potassium, sodium, triethanolamine (2,2′,2″-nitrilo-tris(ethanol)), tromethamine or zinc salt of elafibranor or GFT1007. In a further particular embodiment, the salt of elafibranor or GFT1007 is selected from a tromethamine, potassium, sodium, L-arginine, benethamine, benzathine, ethanolamine, meglumine, glycine, erbumine, L-lysine, choline, epolamine, magnesium or 2-amino-2-methyl-propan-1-ol salt of elafibranor or GFT1007.

In a preferred embodiment, the pharmaceutical composition of the invention comprises elafibranor or a pharmaceutically acceptable salt thereof.

In a most preferred embodiment, the pharmaceutical composition of the invention comprises elafibranor.

Pharmaceutical composition used in the invention can comprise one or several excipients or vehicles, acceptable within a pharmaceutical context (e.g. saline solutions, physiological solutions, isotonic solutions, etc., compatible with pharmaceutical usage and well-known by one of ordinary skill in the art). This composition can also comprise one or several agents or vehicles chosen among dispersants, solubilisers, stabilisers, preservatives, etc. Agents or vehicles useful for these formulations (liquid and/or injectable and/or solid) are particularly methylcellulose, hydroxymethylcellulose, carboxymethylcellulose, polysorbate 80, mannitol, gelatin, lactose, vegetable oils, acacia, liposomes, etc. Elafibranor or GFT1007 can be formulated for enteral or parenteral administration. For example, elafibranor or GFT1007 can be formulated for oral, intravascular (e.g. intravenous or intra-arterial), intramuscular, intraperitoneal, subcutaneous, transdermal or nasal administration. The pharmaceutical composition can be a solid or liquid dosage form. Illustrative formulations include, without limitation, an injectable suspension, or suspension for oral ingestion, a gel, an oil, a pill, a tablet, a suppository, a powder, a gel cap, a capsule, an aerosol, an ointment, a cream, a patch, or means of galenic forms for a prolonged and/or slow release.

In a preferred embodiment, the pharmaceutical composition of the invention is administered orally. Preferably, the composition is formulated in a form selected from the group consisting of a gel, an oil, a pill, a tablet, a powder, a gel cap, a capsule, and a galenic form or device assuring a prolonged and/or slow release.

In a preferred embodiment, the pharmaceutical composition of the invention is formulated in a tablet.

As used herein, the term “therapeutically effective amount” refers to a quantity of elafibranor or GFT1007 which prevents, removes or reduces PBC and one of its adverse events, in particular a quantity of elafibranor or GFT1007 which prevents, removes or reduces PBC. In particular, the amount of pharmaceutical salt of elafibranor or GFT1007 is intended as the amount of free form of elafibranor or GFT1007 in this pharmaceutical salt.

The quantity to be administered can be adapted by a person skilled in the art. In particular, doses and regimen of administration may be function of the stage and of the severity of PBC to be treated, as well as of the weight, the age and the global health of the subject to be treated, as well as of the judgment of the doctor.

In a particular embodiment, elafibranor, GFT1007, or a pharmaceutically acceptable salt of elafibranor or of GFT1007 is administered at a dose varying between 10 mg and 200 mg per administration, preferentially between 80 mg and 120 mg per administration. In a further particular embodiment, elafibranor, GFT1007, or a pharmaceutically acceptable salt of elafibranor or of GFT1007 is administered at a dose of 80 mg per administration. In another particular embodiment, elafibranor, GFT1007 or a pharmaceutically acceptable salt of elafibranor or of GFT1007 is administered at a dose of 120 mg per administration.

Preferably, the pharmaceutical composition of the invention is administered once a day, in particular is orally administered once a day.

According to an embodiment, the pharmaceutical composition is a solid dosage form, such as a tablet. In a further particular embodiment, said tablet comprises between 10 mg and 200 mg of elafibranor, GFT1007, or of a pharmaceutically acceptable salt of elafibranor or of GFT1007, such as between 80 mg and 120 mg of elafibranor, GFT1007, or of a pharmaceutical salt of elafibranor or of GFT1007. For example, a tablet may comprise 80 mg of elafibranor or GFT1007 or a pharmaceutical salt of elafibranor or of GFT1007 or 120 mg of elafibranor or GFT1007 or a pharmaceutical salt of elafibranor or of GFT1007.

In yet another embodiment, a tablet comprising 80 mg of elafibranor or GFT1007 is orally administered once a day.

In yet another embodiment, a tablet comprising 120 mg of elafibranor or GFT1007 is orally administered once a day.

As used herein, the terms “patient”, “subject” or “individual” are interchangeable and refer to a human, including adult and child. In the context of the invention, the patient is suffering from PBC.

In a particular embodiment, the patient has PBC and responds at least partly to ursodeoxycholic acid (UDCA). In another embodiment, the subject having PBC has an inadequate response to ursodeoxycholic acid (UDCA).

In a particular embodiment, the invention relates to the use of the pharmaceutical composition comprising a compound selected from elafibranor, GFT1007, and a pharmaceutically acceptable salt of elafibranor or GFT1007, in combination with at least one other therapeutically active agent, in the method for treating primary biliary cholangitis (PBC) in a subject having primary biliary cholangitis (PBC) with a baseline alkaline phosphatase (ALP) level more than 1.67×ULN and at most 3×ULN, and an inadequate response to ursodeoxycholic acid. The other active agent may in particular be selected from other anti-cholestatic agents such as UDCA or OCA. The invention thus also relates to the combination of the pharmaceutical composition of the invention with UDCA or OCA, preferably UDCA. In particular, the method comprises administering elafibranor at a dose of 80 mg per administration and ursodeoxycholic acid (UDCA), to the subject in need thereof. More particularly, the method comprises administering elafibranor at a dose of 80 mg per administration and ursodeoxycholic acid (UDCA), to a subject having primary biliary cholangitis (PBC) and an inadequate response to ursodeoxycholic acid. Even more particularly, the method comprises administering elafibranor at a dose of 80 mg per day and UDCA at a dose of 13-15 mg/kg/day, to a subject having PBC and an inadequate response to ursodeoxycholic acid.

### Examples

### NCT04526665 Clinical Trial (ELATIVE®)

Participants aged 18 to 75 years, who had been diagnosed with primary biliary cholangitis according to established criteria, were recruited at 82 sites in 14 countries. Inclusion criteria were an alkaline phosphatase level of ≥1.67 times the upper limit of the normal range (ULN), and a total bilirubin level of ≤2 times the ULN. Prior to screening, eligible participants had received 13 to 15 mg/kg ursodeoxycholic acid treatment per day for at least 12 months (stable dose for at least 3 months) or were intolerant to ursodeoxycholic acid (no treatment for at least 3 months).

In this double-blind, placebo-controlled, phase 3 trial, participants with an inadequate response or intolerance to ursodeoxycholic acid were randomized 2:1 to receive elafibranor 80 mg or placebo once daily. Participants receiving stable doses of ursodeoxycholic acid for at least three months prior to enrollment continued their treatment regimen throughout the trial. The overall double-blind treatment period consisted of two parts. In part one, all randomized participants received a minimum of 52 weeks of double-blind treatment. In part two, participants continued to receive double-blind treatment beyond Week 52 in a variable treatment period until all participants had completed their Week 52 assessment or until a maximum treatment duration of 104 weeks, whichever came first. The first database lock occurred after the last participant completed their Week 52 visit in the double-blind period. At the end of the double-blind period (lasting 52 to 104 weeks), participants could optionally enter an open-label extension where they would receive elafibranor 80 mg for up to five years.

The primary endpoint of Study NCT04526665 was response to treatment in the intent-to-treat population at Week 52, defined as ALP<1.67×ULN, TB≤ULN, and ALP decrease ≥15% from baseline. The primary endpoint was also analyzed according to baseline alkaline phosphatase levels of ≤3 times versus >3 times the ULN.

From September 2020 through May 2023, 161 participants were randomly assigned to receive elafibranor 80 mg (108 participants) or placebo (53 participants); these participants made up the intent-to-treat (ITT) and safety populations.

As required by eligibility criteria, all participants had ALP levels ≥1.67×ULN during screening. At baseline, 96.3% of participants had ALP levels ≥1.67×ULN (97.2% and 94.3% of participants in the elafibranor 80 mg group and placebo groups, respectively) and 39.1% of participants had ALP levels >3×ULN (39.8% and 37.7% in the elafibranor 80 mg and placebo groups, respectively).

### Response for All Participants

Complete normalization of alkaline phosphatase at Week 52 occurred in 15% and 0% of patients receiving elafibranor and placebo, respectively (difference, 15%; odds ratio, infinity; 95% CI, 2.8 to infinity; p=0.0019) (see FIG. 1).

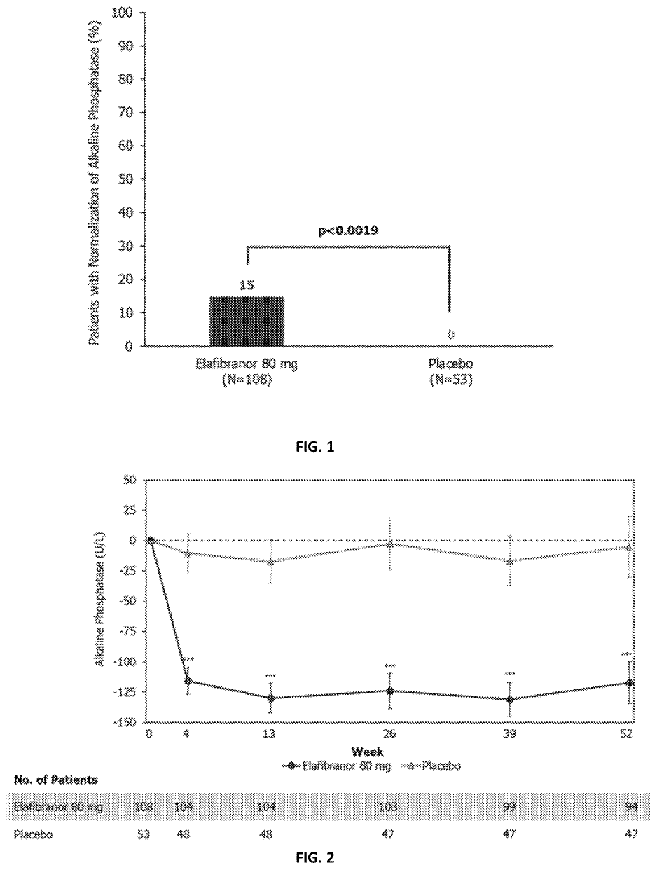

Rapid reductions in alkaline phosphatase levels were observed from baseline through Week 4 in the elafibranor group, and were sustained at levels significantly lower than the placebo group through Week 52 (see FIG. 2).

Response for Participants with Baseline ALP Levels ≤3×ULN or >3×ULN at Week 52

Among participants receiving elafibranor with baseline alkaline phosphatase levels ≤3 times the ULN (65 participants) and >3 times the ULN (43 participants), 70.8% and 20.9% met the primary endpoint, respectively. By contrast, among participants in the placebo group with baseline alkaline phosphatase levels ≤3 times the ULN (33 patients) or >3 times the ULN (20 patients), 6.1% and 0% met the primary endpoint. Complete normalization of alkaline phosphatase at Week 52 occurred in 21.5% and 4.7% of patients receiving elafibranor with baseline alkaline phosphatase levels ≤3 times the ULN and >3 times the ULN, respectively (see Table 1).

TABLE 1
ALP Response by Subgroup at Week 52 (ITT Analysis Set)

| | Characteristic | | | |
|---|---|---|---|---|
| | Elafibranor | Placebo | Elafibranor | Placebo |
| | (N = 108) | (N = 53) | (N = 108) | (N = 53) |
| Analysis Set | 1.67 ULN < ALP ≤ 3 ULN | | ALP > 3 ULN | |
| n | 65 | 33 | 43 | 20 |
| Primary | 46 (70.8) | 2 (6.1) | 9 (20.9) | 0 (0) |
| endpoint met | | | | |
| at week 52, n | | | | |
| (%) | | | | |

Consequently, the cholestatic response rate was lower in participants with higher baseline ALP values >3×ULN (20.9%; none with placebo) compared to participants with lower baseline ALP values ≤3×ULN (70.8% versus 6.1%). Elafibranor has shown efficacy for treating primary biliary cholangitis (PBC) in a subject having PBC with a baseline plasma alkaline phosphatase (ALP) level of more than 1.67×ULN and at most 3×ULN.

Response for Participants with Baseline ALP<3×ULN, AST<2×ULN and TB≤1 mg/dl (Paris I)

TABLE 2
Proportion of patients with response to treatment according
to Paris I criteria at week 52 (ITT Analysis Set)

| | Characteristic | |
|---|---|---|
| | Elafibranor | Placebo |
| | (N = 108) | (N = 53) |
| Analysis Set | Response according to Paris I criteria | |
| | (ALP <3 × ULN, AST <2 × ULN and | |
| | TB ≤1 mg/dL) | |
| n (missing) | 106 (2) | 53 (0) |
| Primary endpoint met at | 73 (68.9) | 25 (47.2) |
| week 52, n (%) | | |
| Proportion of responders | 0.689 | 0.472 |
| 95% CI | [0.595; 0.769] | [0.344; 0.603] |
| Risk difference | 0.220 | — |
| 95% CI | [0.057; 0.374] | |
| Odds Ratio of response | 2.852 | — |
| (elafibranor vs placebo) | [1.339; 7.181] | |
| p value | 0.0061 | — |

Using the ITT Analysis Set, a greater proportion of participants treated with elafibranor demonstrated a response to treatment based on ALP<3×ULN, AST<2×ULN, and TB≤1 mg/dl (Paris I) at Week 52 compared with participants who received placebo. In the elafibranor group 73/106 (68.9%) participants demonstrated a response compared with 25/53 (47.2%) participants in the placebo group, resulting in a difference of 22.0% (95% CI: 5.7; 37.4); the odds ratio of response (elafibranor versus placebo) was 2.852 ([95% CI: 1.339; 7.181]; p=0.0061) (Table 2).

Response for Participants with Baseline ALP Levels ≤2, 2-≤2.5, 2.5-≤3, 3-≤4, and >4×ULN at Week 52

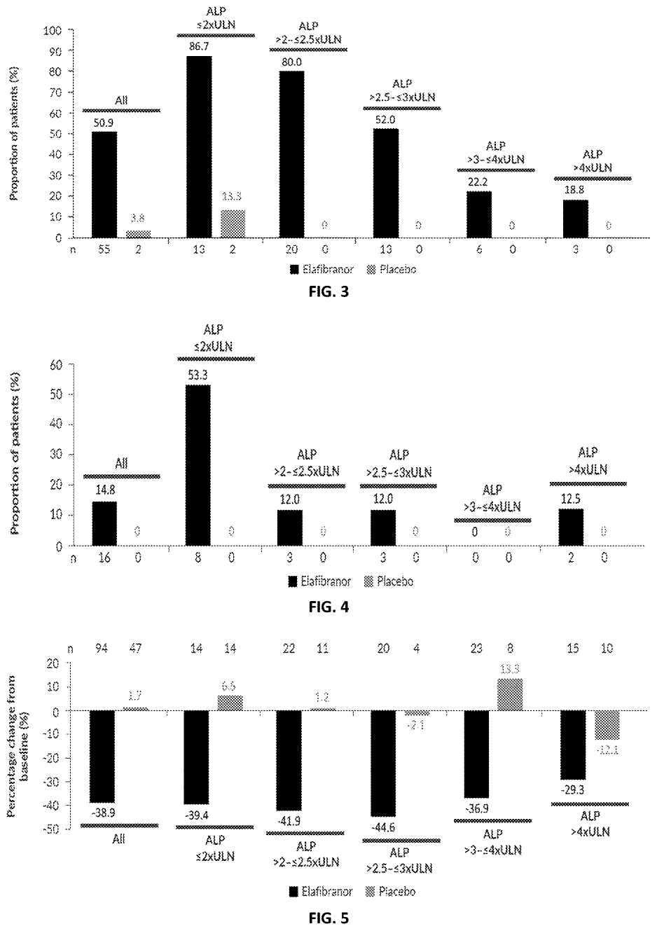

FIG. 3 shows the percentage of participants receiving elafibranor with a biochemical response at Week 52 stratified by baseline ALP levels. Biochemical response (the primary endpoint) was defined as ALP<1.67×ULN, with a ≥15% reduction from baseline, and normal TB.

FIG. 4 shows the percentage of participants receiving elafibranor with ALP normalization at Week 52 stratified by baseline ALP levels.

FIG. 5 shows the mean percentage change from baseline to Week 52 in ALP stratified by baseline ALP levels.

FIGS. 3 to 5 show that participants with lower baseline ALP levels (≤3 ULN) and receiving elafibranor, had higher biochemical response and ALP normalization rates versus those with higher baseline ALP level.

In particular:
FIG. 3 shows that more than 80% of participants receiving elafibranor with baseline ALP levels between more than 1.67×ULN and at most 2.5×ULN, had a biochemical response at Week 52;
FIG. 3 also shows that more than 50% of participants receiving elafibranor with baseline ALP levels between more than 2.5×ULN and at most 3×ULN, had a biochemical response at Week 52, whereas this biochemical response is observed in less than 23% of participants receiving elafibranor with baseline ALP levels of more than 3×ULN; and
FIG. 4 shows that ALP normalization is at least 4 times more frequent in participants receiving elafibranor with baseline ALP levels between more than 1.67×ULN and at most 2×ULN than others participants,

1. A method for treating primary biliary cholangitis (PBC) in a subject having PBC with a baseline plasma alkaline phosphatase (ALP) level of more than 1.67×ULN and at most 3×ULN, the method comprising administering to the subject a therapeutically effective amount of a pharmaceutical composition comprising a compound selected from the group consisting of elafibranor, 2-[2,6-dimethyl-4-[3-[4-(methylthio)phenyl]-3-oxo-propyl]phenoxy]-2-methylpropanoic acid (GFT1007), and a pharmaceutically acceptable salt of elafibranor or GFT1007.

2. The method of claim 1, wherein the pharmaceutical composition comprises elafibranor or a pharmaceutically acceptable salt thereof.

3. The method of claim 2, wherein the pharmaceutical composition comprises elafibranor.

4. The method of claim 1, wherein the pharmaceutical composition is formulated in a form selected from the group consisting of a gel, an oil, a pill, a tablet, a powder, a gel cap, a capsule, and a galenic form or device assuring a prolonged and/or slow release.

5. The method of claim 4, wherein the pharmaceutical composition is formulated in the form of a tablet.

6. The method of claim 3, wherein the pharmaceutical composition is administered once a day.

7. The method of claim 1, wherein the administration is oral.

8. The method of claim 1, wherein the therapeutically effective amount of the pharmaceutical composition contains 80 mg or 120 mg of the compound per administration.

9. The method of claim 8, wherein the therapeutically effective amount of the pharmaceutical composition contains 80 mg of elafibranor per administration.

10. The method of claim 1, wherein the subject has an inadequate response to ursodeoxycholic acid (UDCA).

11. The method of claim 1, further comprising administering ursodeoxycholic acid (UDCA) to the subject.

12. The method of claim 11, wherein the pharmaceutical composition comprises elafibranor at a dose of 80 mg per administration.

13. The method of claim 12, wherein the subject has an inadequate response to UDCA.

14. A method for reducing baseline plasma ALP level in a subject having PBC with a baseline plasma ALP level of more than 1.67×ULN and at most 3×ULN, the method comprising administering to said subject a therapeutically effective amount of a pharmaceutical composition comprising a compound selected from the group consisting of elafibranor, GFT1007, and a pharmaceutically acceptable salt of elafibranor or GFT1007.

15. The method of claim 14, wherein the pharmaceutical composition comprises elafibranor or a pharmaceutically acceptable salt thereof.

16. The method of claim 15, wherein the pharmaceutical composition comprises elafibranor.

17. The method of claim 14, wherein the pharmaceutical composition is formulated in a form selected from the group consisting of a gel, an oil, a pill, a tablet, a powder, a gel cap, a capsule, and a galenic form or device assuring a prolonged and/or slow release.

18. The method of claim 17, wherein the pharmaceutical composition is formulated in the form of a tablet.

19. The method of claim 16, wherein the pharmaceutical composition is administered once a day.

20. The method of claim 14, wherein the administration is oral.

21. The method of claim 14, wherein the therapeutically effective amount of the pharmaceutical composition contains 80 mg or 120 mg of the compound per administration.

22. The method of claim 21, wherein the therapeutically effective amount of the pharmaceutical composition contains 80 mg of elafibranor per administration.

23. The method of claim 14, wherein the subject has an inadequate response to ursodeoxycholic acid (UDCA).

24. The method of claim 14, further comprising administering ursodeoxycholic acid (UDCA) to the subject.
